(19) United States
(12) Patent Application Publication
(10) Pub. No.: US 2009/0289248 A1
(43) Pub. Date: Nov. 26, 2009

(54) DIOXAANTHANTHRENE COMPOUND AND SEMICONDUCTOR DEVICE

(75) Inventors: Norihito Kobayashi, Kanagawa (JP); Mari Sasaki, Kanagawa (JP)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: SONY CORPORATION, Tokyo (JP)

(21) Appl. No.: 12/469,343

(22) Filed: May 20, 2009

(30) Foreign Application Priority Data
May 26, 2008 (JP) 2008-136292

(51) Int. Cl. H01L 51/30; C07D 311/78

(52) U.S. Cl. 257/40; 549/381; 257/E51.041

(57) ABSTRACT

A dioxaanthanthrene compound is represented by structural formula (1):

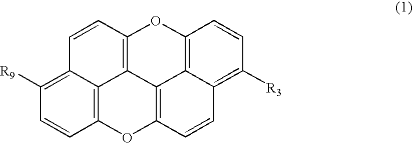

wherein at least one of R₃ and R₉ represents a substituent other than hydrogen.

### BACKGROUND OF THE INVENTION

1. Field of the Invention

The present invention relates to dioxaanthanthrene compounds and semiconductor devices including semiconductor layers composed of such dioxaanthanthrene compounds.

2. Description of the Related Art

In recent years, semiconductor devices including semiconductor layers composed of organic semiconductor materials have been receiving considerable attention. In such semiconductor devices, semiconductor layers can be formed by low-temperature coating in contrast to structures including semiconductor layers composed of inorganic materials. Therefore, such semiconductor devices are advantageous in that device area can be increased, and can be disposed on a flexible substrate that has low heat resistance, such as a plastic substrate. An increase in the range of functions and a reduction in cost are also expected.

As organic semiconductor materials constituting semiconductor layers, for example, polyacenes, such as anthracene, naphthacene, and pentacene, the structural formulae of which are shown below, have been widely researched to date.

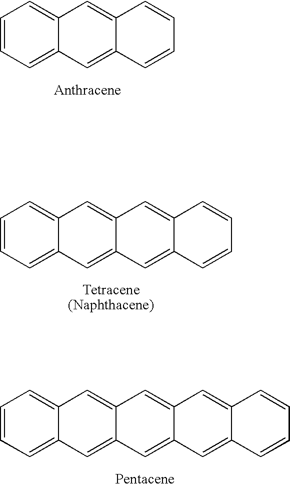

These acene compounds have high crystallinity because of strong cohesion resulting from the intermolecular interactions utilizing the “C—H . . . pi” interactions between adjacent molecules. Here, the “C—H . . . pi” interaction is one of the interactions acting between two adjacent molecules and refers to the state in which the C—H groups (edges) in the periphery of a molecule are weakly attracted toward the pi orbital (faces) above and below the molecular plane, generally resulting in an edge-to-face arrangement. In the solid state, the molecules pack in a herringbone arrangement in which the molecules are in contact with each other at planes and sides. It has been reported that such an arrangement provides high carrier mobility and exhibits excellent semiconductor device properties (refer to Wei-Qiao Deng and William A. Goddard III, J. Phys. Chem. B, 2004 American Chemical Society, Vol. 108, No. 25, 2004, p. 8614-8621).

However, in general, it is considered that the herringbone packing arrangement is disadvantageous to carrier conduction in view of overlapping of molecular orbitals when compared to packing in the pi-stacking arrangement in which molecules are stacked such that the molecular planes are arranged in parallel. Accordingly, a method has been proposed in which the herringbone packing arrangement is prevented by introducing bulky substituents into the pentacene skeleton, and the pentacene backbones responsible for carrier conduction are allowed to pack in a pi-stacking arrangement as shown in FIG. 7 (refer to U.S. Pat. No. 6,690,029 B1).

### SUMMARY OF THE INVENTION

However, in order to achieve packing of pentacene backbones in the pi-stacking arrangement, it is necessary to introduce bulky substituents as described above, which results in low freedom of molecular design. Therefore, it is difficult, for example, to precisely adjust the physical properties according to processes.

Furthermore, with respect to peri-xanthenoxanthenes, a method of producing the peri-xanthenoxanthene molecules themselves has been reported by Pummerer et al. (refer to Ber. Dtsch. Chem. Ges., 59, 2159, 1926). Furthermore, it has been known that the molecules pack in the pi-stacking arrangement in the neutral state in the absence of an applied voltage and in the ionic state in the presence of an applied voltage (refer to Asari, et al., Bull. Chem. Soc. Jpn., 74, 53, 2001). Furthermore, peri-xanthenoxanthene derivatives have been reported by A. E. Wetherby Jr., et al. (refer to Inorg. Chim. Acta., 360, 1977, 2007). Such peri-xanthenoxanthene derivatives have bulky substituents, and are completely different from dioxaanthanthrene compounds according to the embodiments of the present invention which will be described later.

It is desirable to provide an organic semiconductor material (specifically, a dioxaanthanthrene compound) which provides high carrier mobility and high freedom of molecular design and which has excellent adaptability to processes, and to provide a semiconductor device including a semiconductor layer composed of such an organic semiconductor material (specifically, a dioxaanthanthrene compound).

A dioxaanthanthrene compound according to a first embodiment of the present invention is represented by structural formula (1) below, wherein at least one of R₃ and R₉ represents a substituent other than hydrogen. In other words, the dioxaanthanthrene compound according to the first embodiment of the present invention is an organic semiconductor material which is obtained by replacement with a substituent other than hydrogen at least one of positions 3 and 9 of 6,12-dioxaanthanthrene (peri-xanthenoxanthene, which may be abbreviated as “PXX”).

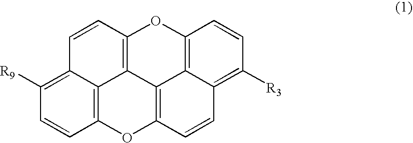

A semiconductor device according to a first embodiment of the present invention includes a gate electrode, a gate insulating layer, source/drain electrodes, and a channel-forming region that are disposed on a base, in which the channel-forming region is composed of the dioxaanthanthrene compound represented by structural formula (1) described above, wherein at least one of R₃ and R₉ represents a substituent other than hydrogen.

A dioxaanthanthrene compound according to a second embodiment of the present invention is represented by structural formula (2) below, wherein at least one of R₁, R₃, R₄, R₅, R₇, R₉, R₁₀, and R₁₁ represents a substituent other than hydrogen. In other words, the dioxaanthanthrene compound according to the second embodiment of the present invention is an organic semiconductor material which is obtained by replacement with a substituent other than hydrogen at least one of positions 1, 3, 4, 5, 7, 9, 10, and 11 of 6,12-dioxaanthanthrene.

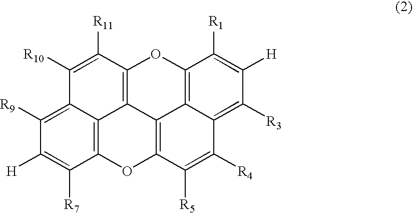

A semiconductor device according to a second embodiment of the present invention includes a gate electrode, a gate insulating layer, source/drain electrodes, and a channel-forming region that are disposed on a base, in which the channel-forming region is composed of the dioxaanthanthrene compound represented by structural formula (2) described above, wherein at least one of R₁, R₃, R₄, R₅, R₇, R₉, R₁₀, and R₁₁ represents a substituent other than hydrogen.

A dioxaanthanthrene compound according to a third embodiment of the present invention includes 6,12-dioxaanthanthrene which is replaced at least one of positions 3 and 9 with a substituent other than hydrogen, the dioxaanthanthrene compound being obtained by halogenating peri-xanthenoxanthene into 3,9-dihalo-peri-xanthenoxanthene and then replacing the halogen atom with the substituent. In this case, the halogen atom may be bromine (Br). In the dioxaanthanthrene compound according to the third embodiment of the present invention including such a case, the substituent may be an aryl group or aryl-alkyl group, may be an aryl group which is replaced at least one of positions 2 to 6 with an alkyl group, or may be an aryl group which is replaced at least one of positions 2 to 6 with an aryl group. Furthermore, the substituent may be a p-tolyl group, p-ethylphenyl group, p-isopropylphenyl group, 4-propylphenyl group, 4-butylphenyl group, 4-nonylphenyl group, or p-biphenyl.

A dioxaanthanthrene compound according to a fourth embodiment of the present invention includes 3,9-diphenyl-peri-xanthenoxanthene obtained by reacting peri-xanthenoxanthene with bromine to produce 3,9-dibromo-peri-xanthenoxanthene, and then by replacing bromine atoms with phenyl groups.

A dioxaanthanthrene compound according to a fifth embodiment of the present invention includes 3,9-di(trans-1-octen-1-yl)-peri-xanthenoxanthene obtained by reacting peri-xanthenoxanthene with bromine to produce 3,9-dibromo-peri-xanthenoxanthene, and then by replacing bromine atoms with trans-1-octen-1-yl groups.

A dioxaanthanthrene compound according to a sixth embodiment of the present invention includes 3,9-di(2-naphthyl)-peri-xanthenoxanthene obtained by reacting peri-xanthenoxanthene with bromine to produce 3,9-dibromo-peri-xanthenoxanthene, and then by replacing bromine atoms with β-naphthyl groups.

A dioxaanthanthrene compound according to a seventh embodiment of the present invention includes 3,9-bis(2,2′-bithiophen-5-yl)-peri-xanthenoxanthene obtained by reacting peri-xanthenoxanthene with bromine to produce 3,9-dibromo-peri-xanthenoxanthene, and then by replacing bromine atoms with 2,2′-bithiophen-5-yl groups.

A dioxaanthanthrene compound according to an eighth embodiment of the present invention includes 3,9-bis(trans-2-(4-pentylphenyl)vinyl)-peri-xanthenoxanthene obtained by reacting peri-xanthenoxanthene with bromine to produce 3,9-dibromo-peri-xanthenoxanthene, and then by replacing bromine atoms with trans-2-(4-pentylphenyl)vinyl groups.

A dioxaanthanthrene compound according to a ninth embodiment of the present invention includes 3,9-di(p-tolyl)-peri-xanthenoxanthene obtained by reacting peri-xanthenoxanthene with bromine to produce 3,9-dibromo-peri-xanthenoxanthene, and then by replacing bromine atoms with p-tolyl groups.

A dioxaanthanthrene compound according to a tenth embodiment of the present invention includes 3,9-bis(p-ethylphenyl)-peri-xanthenoxanthene obtained by reacting peri-xanthenoxanthene with bromine to produce 3,9-dibromo-peri-xanthenoxanthene, and then by replacing bromine atoms with p-ethylphenyl groups.

A dioxaanthanthrene compound according to an eleventh embodiment of the present invention includes 3,9-bis(p-isopropylphenyl)-peri-xanthenoxanthene obtained by reacting peri-xanthenoxanthene with bromine to produce 3,9-dibromo-peri-xanthenoxanthene, and then by replacing bromine atoms with p-isopropylphenyl groups.

A dioxaanthanthrene compound according to a twelfth embodiment of the present invention includes 3,9-bis(4-propylphenyl)-peri-xanthenoxanthene obtained by reacting peri-xanthenoxanthene with bromine to produce 3,9-dibromo-peri-xanthenoxanthene, and then by replacing bromine atoms with 4-propylphenyl groups.

A dioxaanthanthrene compound according to a thirteenth embodiment of the present invention includes 3,9-bis(4-butylphenyl)-peri-xanthenoxanthene obtained by reacting peri-xanthenoxanthene with bromine to produce 3,9-dibromo-peri-xanthenoxanthene, and then by replacing bromine atoms with 4-butylphenyl groups.

A dioxaanthanthrene compound according to a fourteenth embodiment of the present invention includes 3,9-bis(4-nonylphenyl)-peri-xanthenoxanthene obtained by reacting peri-xanthenoxanthene with bromine to produce 3,9-dibromo-peri-xanthenoxanthene, and then by replacing bromine atoms with 4-nonylphenyl groups.

A dioxaanthanthrene compound according to a fifteenth embodiment of the present invention includes 3,9-bis(p-biphenyl)-peri-xanthenoxanthene obtained by reacting peri-xanthenoxanthene with bromine to produce 3,9-dibromo-peri-xanthenoxanthene, and then by replacing bromine atoms with p-biphenyl groups.

In the dioxaanthanthrene compound according to any of the embodiments of the present invention suitable for forming semiconductor layers, the molecules pack in the pi-stacking arrangement in the neutral state in the absence of an applied voltage and in the ionic state in the presence of an applied voltage. Therefore, without introducing bulky substituents, the backbones of the dioxaanthanthrene compound according to any of the embodiments of the present invention can easily pack in the pi-stacking arrangement. Consequently, it is possible to increase the freedom of molecular design of an organic semiconductor material constituting semiconductor layers that exhibit high carrier mobility, and to facilitate molecular design. Moreover, adaptability to processes can be improved. That is, channel-forming regions can be formed not only by a PVD method but also by a wet process, such as coating or printing. Thereby, it is possible to easily produce high-performance semiconductor devices having high carrier mobility.

### BRIEF DESCRIPTION OF THE DRAWINGS

FIG. 1 is a diagram showing a synthesis scheme of dibromo-peri-xanthenoxanthene;

FIGS. 2A, 2B, and 2C are diagrams respectively showing a molecular structure, a crystal structure, and a stacking structure along the c-axis of 3,9-diphenyl-peri-xanthenoxanthene which is a dioxaanthanthrene compound in Example 1;

FIGS. 3A and 3B are diagrams respectively showing a molecular structure and a crystal structure of 3,9-di(trans-1-octen-1-yl)-peri-xanthenoxanthene which is a dioxaanthanthrene compound in Example 2;

FIG. 4 is a graph showing the gate voltage dependence of the current-voltage curve (I-V characteristics) between source/drain electrodes in a test semiconductor device fabricated using 3,9-diphenyl-peri-xanthenoxanthene which is a dioxaanthanthrene compound in Example 1;

FIG. 5A is a schematic partial sectional view of a bottom gate/top contact type field-effect transistor, and FIG. 5B is a schematic partial sectional view of a bottom gate/bottom contact type field-effect transistor;

FIG. 6A is a schematic partial sectional view of a top gate/top contact type field-effect transistor, and FIG. 6B is a schematic partial sectional view of a top gate/bottom contact type field-effect transistor; and

FIG. 7 is a diagram showing an example of packing in the pi-stacking arrangement.

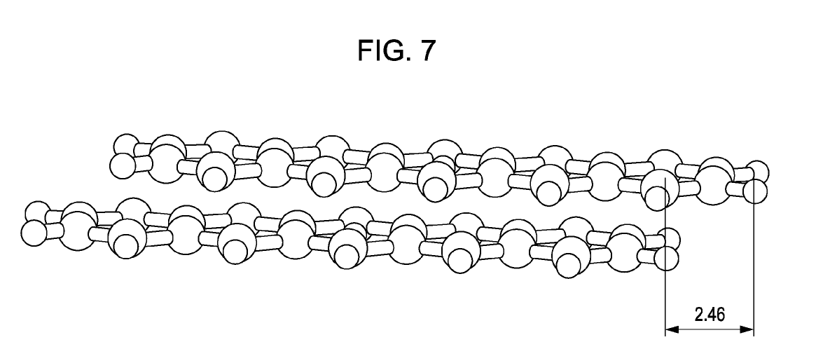

### DESCRIPTION OF THE PREFERRED EMBODIMENTS

The embodiments of the present invention will be described on the basis of examples with reference to the drawings. However, it is to be understood that the present invention is not limited to the examples, and various numerical values and materials in the examples are merely for illustrative purposes. The description will be made in the following order:

1. Overall description on dioxaanthanthrene compounds according to first to fifteenth embodiments of the present invention and semiconductor devices according to first and second embodiments of the present invention
2. Example 1 (Dioxaanthanthrene compounds according to first to fourth embodiments of the present invention)
3. Example 2 (Dioxaanthanthrene compounds according to first to third and fifth embodiments of the present invention)
4. Example 3 (Dioxaanthanthrene compounds according to first to third and sixth embodiments of the present invention)
5. Example 4 (Dioxaanthanthrene compounds according to first to third and seventh embodiments of the present invention)
6. Example 5 (Dioxaanthanthrene compounds according to first to third and eighth embodiments of the present invention)
7. Example 6 (Dioxaanthanthrene compounds according to first to third and ninth embodiments of the present invention)
8. Example 7 (Dioxaanthanthrene compounds according to first to third and tenth embodiments of the present invention)
9. Example 8 (Dioxaanthanthrene compounds according to first to third and eleventh embodiments of the present invention)
10. Example 9 (Dioxaanthanthrene compounds according to first to third and twelfth embodiments of the present invention)
11. Example 10 (Dioxaanthanthrene compounds according to first to third and thirteenth embodiments of the present invention)
12. Example 11 (Dioxaanthanthrene compounds according to first to third and fourteenth embodiments of the present invention)
13. Example 12 (Dioxaanthanthrene compounds according to first to third and fifth embodiments of the present invention)
14. Example 13 (Semiconductor devices according to first and second embodiments of the present invention, and others)

[Overall Description on Dioxaanthanthrene Compounds according to First to Fifteenth Embodiments of the Present Invention and Semiconductor Devices according to First and Second Embodiments of the Present Invention]

In the description below, dioxaanthanthrene compounds according to the first embodiment of the present invention or semiconductor devices according to the first embodiment of the present invention may be collectively simply referred to as the “first embodiment of the present invention”. Furthermore, dioxaanthanthrene compounds according to the second embodiment of the present invention or semiconductor devices according to the second embodiment of the present invention may be collectively simply referred to as the “second embodiment of the present invention”.

The first embodiment of the present invention may include the following cases:

(1-1) Case where R₃ is a substituent other than hydrogen, and R₉ is a hydrogen atom.
(1-2) Case where R₉ is a substituent other than hydrogen, and R₃ is a hydrogen atom.
(1-3) Case where R₃ and R₉ are each a substituent other than hydrogen. In case (1-3), R₃ and R₉ may be the same substituent or different substituents.

The second embodiment of the present invention may include the following cases:

(2-1) Case where R₁ is a substituent other than hydrogen, and R₃ to R₁₁ are each a substituent other than hydrogen or a hydrogen atom (2⁷ cases in total).
(2-2) Case where R₃ is a substituent other than hydrogen, and R₁ and R₄ to R₁₁ are each a substituent other than hydrogen or a hydrogen atom (2⁷ cases in total).
(2-3) Case where R₄ is a substituent other than hydrogen, and R₁, R₃, and R₅ to R₁₁ are each a substituent other than hydrogen or a hydrogen atom (2⁷ cases in total).
(2-4) Case where R₅ is a substituent other than hydrogen, and R₁ to R₄ and R₇ to R₁₁ are each a substituent other than hydrogen or a hydrogen atom (2⁷ cases in total).
(2-5) Case where R₇ is a substituent other than hydrogen, and R₁ to R₅ and R₉ to R₁₁ are each a substituent other than hydrogen or a hydrogen atom (27 cases in total).
(2-6) Case where R₉ is a substituent other than hydrogen, and R₁ to R₇, R₁₀, and R₁₁ are each a substituent other than hydrogen or a hydrogen atom (2⁷ cases in total).
(2-7) Case where R₁₀ is a substituent other than hydrogen, and R₁ to R₉ and R₁₁ are each a substituent other than hydrogen or a hydrogen atom (2⁷ cases in total).
(2-8) Case where R₁₁ is a substituent other than hydrogen, and R₁ to R₁₀ are each a substituent other than hydrogen or a hydrogen atom (2⁷ cases in total).

The cases described above include duplicate cases. R₁, R₃, R₄, R₅, R₇, R₉, R₁₀, and R₁₁ may be the same substituent or different substituents.

In the second embodiment of the present invention, at least one of R₃ and R₉ may be a substituent other than hydrogen, and at least one of R₁, R₄, R₅, R₇, R₁₀ and R₁₁ may be a substituent other than hydrogen. Furthermore, in the second embodiment of the present invention, at least one of R₃ and R₉ may be a substituent other than hydrogen, and at least one of R₄, R₅, R₁₀, and R₁₁ may be a substituent other than hydrogen.

Specifically, for example, such preferred embodiments may include the following cases:

(3-1) Case where R₃ is a substituent other than hydrogen, and R₁ and R₄ to R₁₁ are each a substituent other than hydrogen or a hydrogen atom (2⁷ cases in total).
(3-2) Case where R₉ is a substituent other than hydrogen, and R₁ to R₇, R₁₀, and R₁₁ are each a substituent other than hydrogen or a hydrogen atom (2⁷ cases in total).
(3-3) Case where R₃ and R₉ are each a substituent other than hydrogen, and R₁, R₄, R₅, R₇, R₁₀, and R₁₁ are each a substituent other than hydrogen or a hydrogen atom (2⁶ cases in total).
(3-4) Case where R₃ is a substituent other than hydrogen, R₁ is a hydrogen atom, and R₄ to R₁₁ are each a substituent other than hydrogen or a hydrogen atom (2⁶ cases in total).
(3-5) Case where R₃ is a substituent other than hydrogen, R₇ is a hydrogen atom, and R₁, R₄, R₅, and R₉ to R₁₁ are each a substituent other than hydrogen or a hydrogen atom (2⁶ cases in total).
(3-6) Case where R₉ is a substituent other than hydrogen, R₁ is a hydrogen atom, and R₃ to R₇, R₁₀, and R₁₁ are each a substituent other than hydrogen or a hydrogen atom (2⁶ cases in total).
(3-7) Case where R₉ is a substituent other than hydrogen, R₇ is a hydrogen atom, and R₁, R₃ to R₅, R₁₀, and R₁₁ are each a substituent other than hydrogen or a hydrogen atom (2⁶ cases in total).
(3-8) Case where R₃ and R₉ are each a substituent other than hydrogen, R₁ is a hydrogen atom, and R₄ to R₇, R₁₀, and R₁₁ are each a substituent other than hydrogen or a hydrogen atom (2⁵ cases in total).
(3-9) Case where R₃ and R₉ are each a substituent other than hydrogen, R₇ is a hydrogen atom, and R₁, R₄, R₅, R₁₀, and R₁₁ are each a substituent other than hydrogen or a hydrogen atom (2⁵ cases in total).
(3-10) Case where R₃ and R₉ are each a substituent other than hydrogen, R₁ and R₇ are each a hydrogen atom, and R₄, R₅, R₁₀, and R₁₁ are each a substituent other than hydrogen or a hydrogen atom (2⁴ cases in total).

The cases described above include duplicate cases. R₁, R₃, R₄, R₅, R₇, R₉, R₁₀, and R₁₁ may be the same substituent or different substituents.

In the embodiments including the preferred embodiments of the present invention, the substituent other than hydrogen may be a substituent selected from the group consisting of an alkyl group, a cycloalkyl group, an alkenyl group, an alkynyl group, an aryl group, an aryl-alkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorohydrocarbon group, a cyano group, a nitro group, a hydroxy group, a mercapto group, and a silyl group.

Furthermore, in the embodiments including the preferred embodiments of the present invention, the substituent other than hydrogen may be a substituent selected from the group consisting of an alkyl group, an alkenyl group, an aryl group, an aryl-alkyl group, an aromatic heterocycle, and a halogen atom.

Examples of the alkyl group include methyl, ethyl, propyl, isopropyl, tertiary butyl, pentyl, hexyl, octyl, and dodecyl groups, which may be straight-chain or branched. Examples of the cycloalkyl group include cyclopentyl and cyclohexyl groups; examples of the alkenyl group include a vinyl group; examples of the alkynyl group include an ethynyl group; examples of the aryl group include phenyl, naphthyl, and biphenyl groups; examples of the aryl-alkyl group include methylaryl, ethylaryl, isopropylaryl, normal butylaryl, p-tolyl, p-ethylphenyl, p-isopropylphenyl, 4-propylphenyl, 4-butylphenyl, and 4-nonylphenyl groups; examples of the aromatic heterocycle include pyridyl, thienyl, furyl, pyridazinyl, pyrimidinyl, pyrazinyl, triazinyl, imidazolyl, pyrazolyl, thiazolyl, quinazolinyl, and phthalazinyl groups; examples of the heterocyclic group include pyrrolidyl, imidazolidyl, morpholinyl, and oxazolidyl groups; examples of the alkoxy group include methoxy, ethoxy, propyloxy, pentyloxy, and hexyloxy groups; examples of the cycloalkoxy group include cyclopentyloxy and cyclohexyloxy groups; examples of the aryloxy group include phenoxy and naphthyloxy groups; examples of the alkylthio group include methylthio, ethylthio, propylthio, pentylthio, and hexylthio groups; examples of the cycloalkylthio group include cyclopentylthio and cyclohexylthio groups; examples of the arylthio group include phenylthio and naphthylthio groups; examples of the alkoxycarbonyl group include methyloxycarbonyl, ethyloxycarbonyl, butyloxycarbonyl, and octyloxycarbonyl groups; examples of the aryloxycarbonyl group include phenyloxycarbonyl and naphthyloxycarbonyl groups; examples of the sulfamoyl group include aminosulfonyl, methylaminosulfonyl, dimethylaminosulfonyl, cyclohexylaminosulfonyl, phenylaminosulfonyl, naphthylaminosulfonyl, and 2-pyridylaminosulfonyl groups; examples of the acyl group include acetyl, ethylcarbonyl, propylcarbonyl, cyclohexylcarbonyl, octylcarbonyl, 2-ethylhexylcarbonyl, dodecylcarbonyl, phenylcarbonyl, naphthylcarbonyl, and pyridylcarbonyl groups; examples of the acyloxy group include acetyloxy, ethylcarbonyloxy, octylcarbonyloxy, and phenylcarbonyloxy groups; examples of the amide group include methylcarbonylamino, ethylcarbonylamino, dimethylcarbonylamino, pentylcarbonylamino, cyclohexylcarbonylamino, 2-ethylhexylcarbonylamino, phenylcarbonylamino, and naphthylcarbonylamino groups; examples of the carbamoyl group include aminocarbonyl, methylaminocarbonyl, dimethylaminocarbonyl, cyclohexylaminocarbonyl, 2-ethylhexylaminocarbonyl, phenylaminocarbonyl, naphthylaminocarbonyl, and 2-pyridylaminocarbonyl groups; examples of the ureido group include methylureido, ethylureido, cyclohexylureido, dodecylureido, phenylureido, naphthylureido, and 2-pyridylaminoureido groups; examples of the sulfinyl group include methylsulfinyl, ethylsulfinyl, butylsulfinyl, cyclohexylsulfinyl, 2-ethylhexylsulfinyl, phenylsulfinyl, naphthylsulfinyl, and 2-pyridylsulfinyl groups; examples of the alkylsulfonyl group include methylsulfonyl, ethylsulfonyl, butylsulfonyl, cyclohexylsulfonyl, 2-ethylhexylsulfonyl, and dodecylsulfonyl groups; examples of the arylsulfonyl group include phenylsulfonyl, naphthylsulfonyl, and 2-pyridylsulfonyl groups; examples of the amino group include amino, ethylamino, dimethylamino, butylamino, 2-ethylhexylamino, anilino, naphthylamino, and 2-pyridylamino groups; examples of the halogen atom include fluorine, chlorine, bromine, and iodine atoms; and examples of the fluorohydrocarbon group include fluoromethyl, trifluoromethyl, pentafluoroethyl, and pentafluorophenyl groups. Other examples of the substituent include cyano, nitro, hydroxy, and mercapto groups. Examples of the silyl group include trimethylsilyl, triisopropylsilyl, triphenylsilyl, and phenyldiethylsilyl groups. These substituents may be further replaced with another substituent described above. Moreover, a plurality of substituents may be combined together to form a ring.

In a semiconductor device including a gate electrode, a gate insulating layer, source/drain electrodes, and a channel-forming region that are disposed on a base, the channel-forming region may be composed of any of the dioxaanthanthrene compounds according to the third to fifteenth embodiments of the present invention described above. Furthermore, such a semiconductor device can also be configured as any of the bottom gate/bottom contact type field-effect transistor (FET), the bottom gate/top contact type FET, the top gate/bottom contact type FET, and the top gate/top contact type FET which will be described below.

In the case where the semiconductor device according to the first or second embodiment of the present invention is configured as a bottom gate/bottom contact type field-effect transistor (FET), the bottom gate/bottom contact type FET includes (A) a gate electrode disposed on a base, (B) a gate insulating layer disposed on the gate electrode, (C) source/drain electrodes disposed on the gate insulating layer, and (D) a channel-forming region disposed between the source/drain electrodes and on the gate insulating layer.

Furthermore, in the case where the semiconductor device according to the first or second embodiment of the present invention is configured as a bottom gate/top contact type FET, the bottom gate/top contact type FET includes (A) a gate electrode disposed on a base, (B) a gate insulating layer disposed on the gate electrode, (C) a channel-forming region and a channel-forming region extension disposed on the gate insulating layer, and (D) source/drain electrodes disposed on the channel-forming region extension.

Furthermore, in the case where the semiconductor device according to the first or second embodiment of the present invention is configured as a top gate/bottom contact type FET, the top gate/bottom contact type FET includes (A) source/drain electrodes disposed on a base, (B) a channel-forming region disposed between the source/drain electrodes and on the base, (C) a gate insulating layer disposed on the channel-forming region, and (D) a gate electrode disposed on the gate insulating layer.

Furthermore, in the case where the semiconductor device according to the first or second embodiment of the present invention is configured as a top gate/top contact type FET, the top gate/top contact type FET includes (A) a channel-forming region and a channel-forming region extension disposed on a base, (B) source/drain electrodes disposed on the channel-forming region extension, (C) a gate insulating layer disposed on the source/drain electrodes and the channel-forming region, and (D) a gate electrode disposed on the gate insulating layer.

The base can be composed of a silicon oxide-based material, such as SiO_{X} or spin-on glass (SOG); silicon nitride (SiN_{Y}); aluminum oxide (Al₂O₃); or a metal oxide high-dielectric-constant insulating film. When the base is composed of such a material, the base may be formed on (or above) a support composed of any of the materials described below. That is, examples of the material for the support and/or a base other than the base described above include organic polymers, such as polymethyl methacrylate (PMMA), polyvinyl alcohol (PVA), polyvinyl phenol (PVP), polyethersulfone (PES), polyimide, polycarbonate, polyethylene terephthalate (PET), and polyethylene naphthalate (PEN); and mica. When organic polymers are used, the polymeric materials are formed into plastic films, plastic sheets, and plastic substrates having flexibility. By using a base composed of any of such flexible polymeric materials, for example, the resulting field-effect transistor can be built in or integrated into a display device or electronic apparatus having curved surfaces. Other examples of the base include various glass substrates, various glass substrates provided with insulating films on the surfaces thereof, quartz substrates, quartz substrates provided with insulating films on the surfaces thereof, silicon substrates provided with insulating films on the surfaces thereof, and metal substrates composed of various alloys or various metals, such as stainless steel. As a support having electrical insulating properties, an appropriate material may be selected from the materials described above. Other examples of the support include conductive substrates, such as a substrate composed of a metal (e.g., gold), a substrate composed of highly oriented graphite, and a stainless steel substrate. Furthermore, depending on the configuration and structure of the semiconductor device, the semiconductor device may be provided on a support. Such a support can be composed of any of the materials described above.

Examples of the material constituting the gate electrode, source/drain electrodes, and interconnect lines include metals, such as platinum (Pt), gold (Au), palladium (Pd), chromium (Cr), molybdenum (Mo), nickel (Ni), aluminum (Al), silver (Ag), tantalum (Ta), tungsten (W), copper (Cu), titanium (Ti), indium (In), and tin (Sn), alloys containing these metal elements, conductive particles composed of these metals, conductive particles composed of alloys containing these metals, and conductive materials, such as impurity-containing polysilicon. A stacked structure including layers containing these elements may be employed. Furthermore, as the material constituting the gate electrode, source/drain electrodes, and interconnect lines, an organic material (conductive polymer), such as poly(3,4-ethylenedioxythiophene)/polystyrene sulfonic acid [PEDOT/PSS], may be mentioned. The materials constituting the gate electrode, source/drain electrodes, and interconnect lines may be the same or different.

Examples of the method for forming the gate electrode, source/drain electrodes, and interconnect lines include, although depending on the materials constituting them, physical vapor deposition (PVD) methods; various chemical vapor deposition (CVD) methods, such as MOCVD; spin coating methods; various printing methods, such as screen printing, ink-jet printing, offset printing, reverse offset printing, gravure printing, and microcontact printing; various coating methods, such as air-doctor coating, blade coating, rod coating, knife coating, squeeze coating, reverse roll coating, transfer roll coating, gravure coating, kiss coating, cast coating, spray coating, slit orifice coating, calender coating, and dipping; stamping methods; lift-off methods; shadow-mask methods; plating methods, such as electrolytic plating, electroless plating, or a combination of both; and spraying methods. As necessary, these methods may be combined with patterning techniques. Furthermore, examples of PVD methods include (a) various vacuum deposition methods, such as electron beam heating, resistance heating, flash vapor deposition, and crucible heating; (b) plasma deposition methods; (c) various sputtering methods, such as diode sputtering, DC sputtering, DC magnetron sputtering, RF sputtering, magnetron sputtering, ion beam sputtering, and bias sputtering; and (d) various ion plating methods, such as a direct current (DC) method, an RF method, a multi-cathode method, an activation reaction method, an electric field deposition method, an RF ion plating method, and a reactive ion plating method.

Furthermore, examples of the material constituting the gate insulating layer include inorganic insulating materials, such as silicon oxide-based materials, silicon nitride (SiN_{Y}), and metal oxide high-dielectric-constant insulating films; and organic insulating materials, such as polymethyl methacrylate (PMMA), polyvinyl phenol (PVP), and polyvinyl alcohol (PVA). These materials may be used in combination. Examples of the silicon oxide-based materials include silicon oxide (SiO_{X}), BPSG, PSG, BSG, AsSG, PbSG, silicon oxynitride (SiON), spin-on glass (SOG), and low-dielectric-constant materials (e.g., polyaryl ethers, cycloperfluoro carbon polymers, benzocyclobutene, cyclic fluorocarbon resins, polytetrafluoroethylene, fluoroaryl ethers, polyfluoroimide, amorphous carbon, and organic SOG).

Furthermore, the gate insulating layer may be formed by oxidizing or nitriding the surface of the gate electrode or by depositing an oxide film or a nitride film on the surface of the gate electrode. As the method for oxidizing the surface of the gate electrode, although depending on the material constituting the gate electrode, for example, an oxidation method using O₂ plasma or an anodic oxidation method may be mentioned. As the method for nitriding the surface of the gate electrode, although depending on the material constituting the gate electrode, for example, a nitriding method using N₂ plasma may be mentioned. Furthermore, for example, when a gate electrode composed of Au is used, a gate insulating layer may be formed in a self-assembling manner on the surface of the gate electrode by coating the surface of the gate electrode with insulating molecules having functional groups capable of forming chemical bonds with the gate electrode, such as linear hydrocarbon molecules with one end being modified with a mercapto group, using a dipping method or the like.

Examples of the method for forming the channel-forming region, or the channel-forming region and the channel-forming region extension include the various PVD methods described above; spin coating methods; various printing methods described above; various coating methods described above; dipping methods; casting methods; and spraying methods. As necessary, additives (e.g., doping materials, such as n-type impurities and p-type impurities) may be added to the dioxaanthanthrene compound according to the first or second embodiment of the present invention.

When the semiconductor devices according to the embodiments of the present invention are applied to or used for display devices or various types of electronic apparatuses, monolithic integrated circuits in which many semiconductor devices are integrated on supports may be fabricated, or the individual semiconductor devices may be separated by cutting to produce discrete components. Furthermore, the semiconductor devices may be sealed with resins.

### Example 1

Example 1 relates to dioxaanthanthrene compounds according to the first to fourth embodiments of the present invention. The dioxaanthanthrene compounds of Example 1 are represented by structural formula (1) below, wherein at least one of R₃ and R₉ represents a substituent other than hydrogen. Alternatively, the dioxaanthanthrene compounds of Example 1 are represented by structural formula (2) below, wherein at least one of R₁, R₃, R₄, R₅, R₇, R₉, R₁₀, and R₁₁ represents a substituent other than hydrogen.

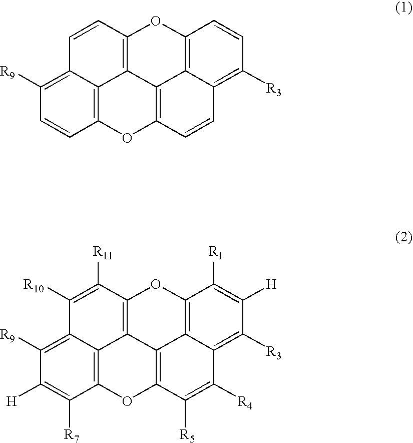

More specifically, a dioxaanthanthrene compound of Example 1 is an organic material which is obtained by replacement with phenyl groups as aryl groups at both of positions 3 and 9 of 6,12-dioxaanthanthrene (PXX), i.e., 3,9-diphenyl-peri-xanthenoxanthene (PXX-Ph₂) represented by structural formula (3) below. That is, R₃ and R₉ are each an aryl group (specifically, phenyl group).

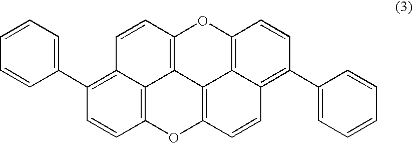

Furthermore, a dioxaanthanthrene compound of Example 1 is 6,12-dioxaanthanthrene which is replaced at least one of positions 3 and 9 with a substituent, the dioxaanthanthrene compound being obtained by halogenating peri-xanthenoxanthene into 3,9-dihalo-peri-xanthenoxanthene and then replacing the halogen atom with the substituent. In this case, specifically, the halogen atom is bromine (Br) The substituent is an aryl group or aryl-alkyl group, or the substituent is an aryl group which is replaced at least one of positions 2 to 6 with an alkyl group or is an aryl group which is replaced at least one of positions 2 to 6 with an aryl group. The same applies to Examples 2 to 12 described later. In Example 1, specifically, the substituent is a phenyl group. Furthermore, a dioxaanthanthrene compound of Example 1 is 3,9-diphenyl-peri-xanthenoxanthene obtained by reacting peri-xanthenoxanthene with bromine to produce 3,9-dibromo-peri-xanthenoxanthene, and then by replacing bromine atoms with phenyl groups.

The PXX-Ph₂ which is the dioxaanthanthrene compound of Example 1 can be synthesized according to the scheme described below.

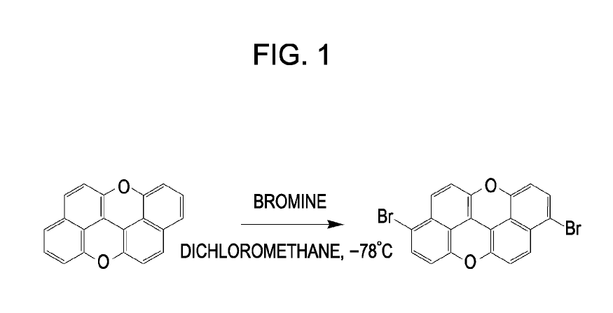

First, PXX-Br₂ which is a bromine substitution product of PXX is synthesized according to the scheme shown in FIG. 1. Specifically, a dichloromethane solution of bromine (2 equivalents) was reacted with a dichloromethane solution of PXX (1 equivalent) at −78° C. Then, the temperature of the reaction mixture was raised to room temperature, and the reaction mixture was treated with an aqueous solution of sodium bisulfite to give a yellow-green crude product. The crude product obtained by filtration was washed with dichloromethane, and thereby 3,9-dibromo-peri-xanthenoxanthene (PXX-Br₂) was obtained. It was confirmed by time-of-flight mass spectrometry (hereinafter abbreviated as “Tof-MS”) and proton nuclear magnetic resonance spectroscopy (¹H-NMR) that this compound was a dibromonated product.

Next, a catalytic amount of tetrakistriphenylphosphine palladium (0) was added to a toluene solution of PXX-Br₂ (1 equivalent) and (4,4,5,5-tetramethyl-1,3,2-dioxaborolan-2-yl)benzene (2 equivalents) in the presence of sodium carbonate, and refluxing was performed for 48 hours. Then, the reaction mixture was left to stand to cool to room temperature and poured into methanol. The resulting precipitated yellow solid was obtained by filtration and washed with methanol, hydrochloric acid, and water. Then, recrystallization was performed from tetrahydrofuran, and thereby yellow needle crystals were obtained. It was confirmed by Tof-MS and ¹H-NMR that the resulting compound was a disubstituted product, i.e., 3,9-diphenyl-peri-xanthenoxanthene (PXX-Ph₂).

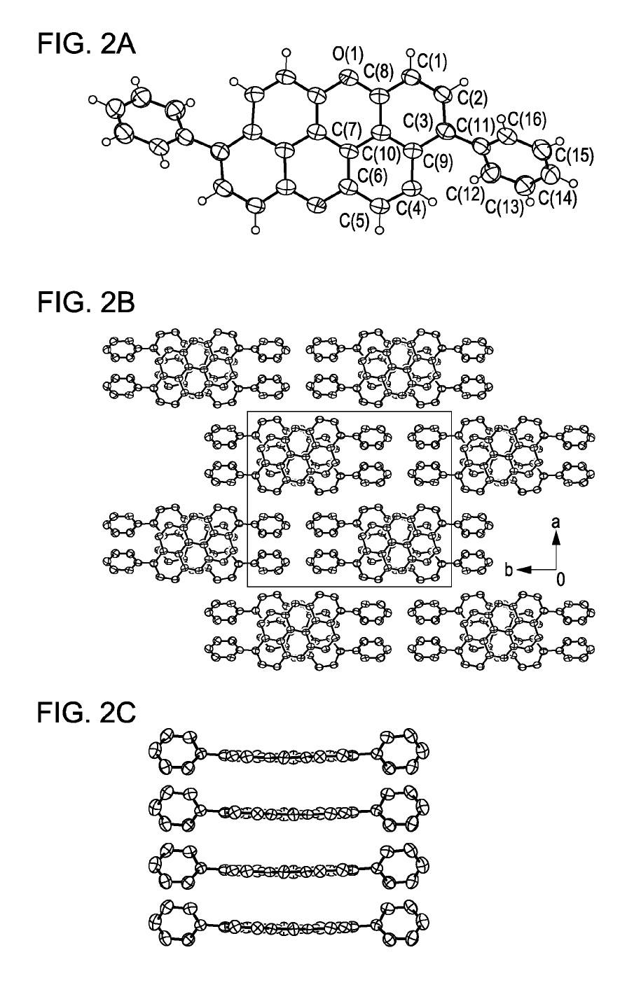

Using the resulting PXX-Ph₂ single crystal, X-ray structural analysis was carried out. The results thereof will be described below. FIG. 2A shows the molecular structure, which confirms that replacement with phenyl groups occurred at positions 3 and 9 of the PXX skeleton. Furthermore, FIG. 2B shows the crystal structure. Adjacent molecules are arranged along the c-axis such that pi-planes of PXX backbones are stacked in parallel (refer to FIG. 2C). The distance in the stacking direction between the molecular planes was 3.47 Å.

Crystal system: Orthorhombic system
Space group: Pccn (#56)

### Lattice Constant

a=15.920(5) Å

b=18.508(5) Å

c=6.930(5) Å

### V=2041.9(17) Å³

### Z=8

In order to evaluate the dioxaanthanthrene compound of Example 1, a test device was fabricated as described below. That is, a silicon semiconductor substrate heavily doped with an n-type dopant and having a thermal oxide film with a thickness of 150 nm on a principal surface thereof was prepared. The surface of the silicon semiconductor substrate was treated with a silane coupling agent. A PXX-Ph₂ thin film with a thickness of 50 nm was formed thereon by a vacuum deposition method. Then, gold electrodes were vapor-deposited, using a metal mask, on the PXX-Ph₂ thin film to form source/drain electrodes. Thereby, a transistor structure was obtained. The silicon semiconductor substrate itself was configured to serve as a gate electrode. The gold electrode pattern serving as the source/drain electrodes includes strip-shaped patterns disposed in parallel, and the distance between the patterns (channel length L) was 50 μm, and the pattern length (channel width W) was 30 mm.

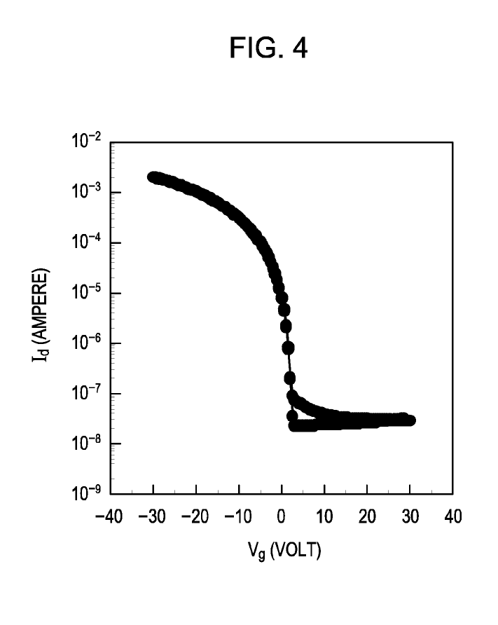

Using the silicon semiconductor substrate as the gate electrode, the gate voltage dependence of the current-voltage curve between source/drain electrodes was measured. The gate voltage was changed from 0 V to −30 V with a step of 10 V. As a result, a drain current saturation phenomenon due to the increase in the drain voltage was confirmed. The hole mobility obtained from the slope of the drain current/gate voltage curve in the saturation region (V_{d}=−40 V) was 0.33 cm²/(V·s). The measurement results are shown in the graph of FIG. 4, in which the horizontal axis represents gate voltage V_{g} (volt), and the vertical axis represents drain current I_{d} (ampere).

For comparison, a test device similar to that described above was fabricated, in which pentacene was used instead of the dioxaanthanthrene compound. Then, as in Example 1, using the silicon semiconductor substrate as the gate electrode, the gate voltage dependence of the current-voltage curve between source/drain electrodes was measured. The gate voltage was changed from 0 V to −30 V with a step of 10 V. The hole mobility obtained from the slope of the drain current/gate voltage curve in the saturation region (V_{d}=−40 V) was 0.2 cm²/(V·s), which was lower than that in Example 1.

### Example 2

Example 2 also relates to dioxaanthanthrene compounds according to the first and second embodiments of the present invention, and further relates to dioxaanthanthrene compounds according to the third and fifth embodiments. A dioxaanthanthrene compound of Example 2 is 3,9-di(trans-1-octen-1-yl)-peri-xanthenoxanthene [PXX-(VC6)₂] represented by structural formula (4) below. That is, R₃ and R₉ each include an alkenyl group (specifically, vinyl group) and an alkyl group.

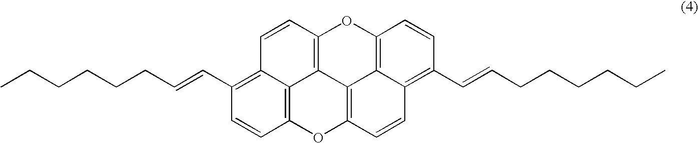

Furthermore, a dioxaanthanthrene compound of Example 2 is 3,9-di(trans-1-octen-1-yl)-peri-xanthenoxanthene obtained by reacting peri-xanthenoxanthene with bromine to produce 3,9-dibromo-peri-xanthenoxanthene, and then by replacing bromine atoms with trans-1-octen-1-yl groups.

PXX-(VC6)₂ of Example 2 was obtained according to the same scheme as that in Example 1, except that 4,4,5,5-tetramethyl-1,3,2-dioxaborolan-2-yl)benzene was changed to trans-1-octen-1-ylboronic acid pinacol ester in the synthesis process. Then, purification was performed by recrystallization from toluene. It was confirmed by Tof-MS and ¹H-NMR that the resulting compound was a disubstituted product, i.e., PXX-(VC6)₂.

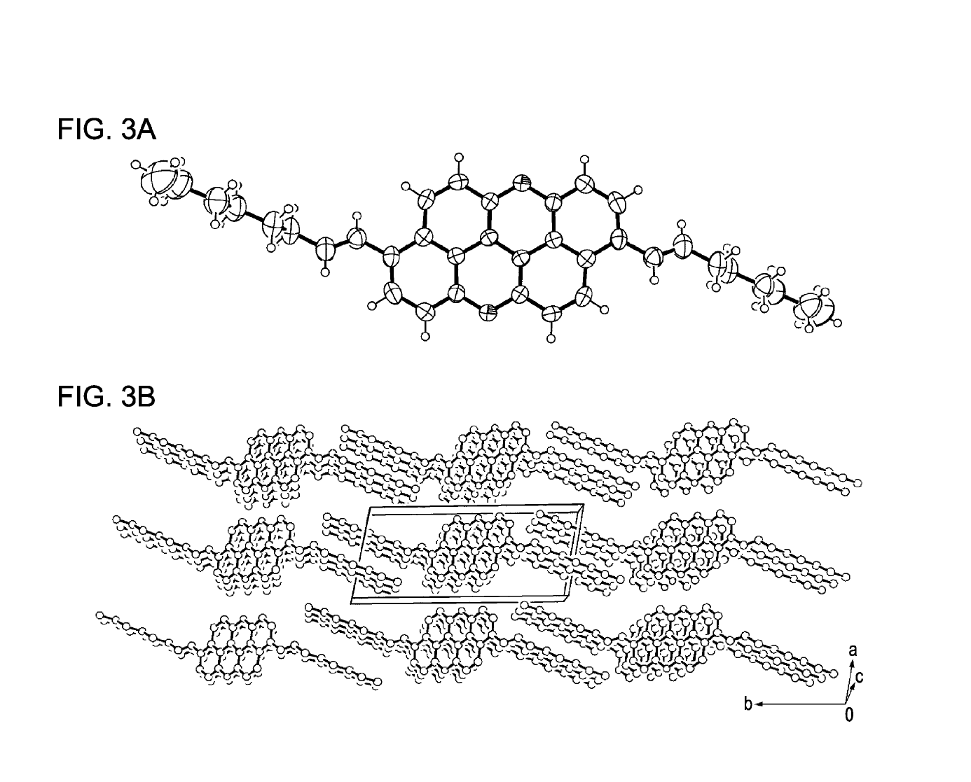

Using the resulting PXX-(VC6)₂ single crystal, X-ray structural analysis was carried out. The results thereof will be described below. FIG. 3A shows the molecular structure, which confirms that replacement with trans-1-octen-1-yl groups occurred at positions 3 and 9 of the PXX skeleton. Furthermore, FIG. 3B shows the crystal structure. Adjacent molecules are arranged along the c-axis such that pi-planes of PXX backbones are stacked in parallel. In the crystal system shown below, the term “P-1” means the following: P 1̅

Crystal system: Triclinic system
Space group: P-1 (#2)

### Lattice Constant

a=8.279(2) Å

b=18.015(5) Å

c=4.9516(13) Å

α=97.291(4)°

β=103.559(4)°

γ=98.867(4)°

### V=699.0(3) Å³

### Z=1

In order to evaluate the dioxaanthanthrene compound of Example 2, a test device was fabricated as in Example 1. In the test device, the gate voltage dependence of the current-voltage curve between source/drain electrodes was measured. The gate voltage was changed from 0 V to −30 V (with a step of 10 V), and as a result, a drain current saturation phenomenon due to the increase in the drain voltage was confirmed. The same applied to Examples 3 to 12 which will be described below.

### Example 3

Example 3 also relates to dioxaanthanthrene compounds according to the first and second embodiments of the present invention, and further relates to dioxaanthanthrene compounds according to the third and sixth embodiments. A dioxaanthanthrene compound of Example 3 is 3,9-di(2-naphthyl)-peri-xanthenoxanthene [PXX-(Nap)₂] represented by structural formula (5) below. That is, R₃ and R₉ are each an aryl group (specifically, β-naphthyl group).

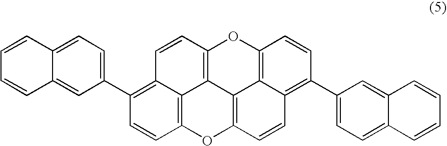

Furthermore, a dioxaanthanthrene compound of Example 3 is 3,9-di(2-naphthyl)-peri-xanthenoxanthene obtained by reacting peri-xanthenoxanthene with bromine to produce 3,9-dibromo-peri-xanthenoxanthene, and then by replacing bromine atoms with β-naphthyl groups.

PXX-(Nap)₂ of Example 3 was obtained according to the same scheme as that in Example 1, except that 4,4,5,5-tetramethyl-1,3,2-dioxaborolan-2-yl)benzene was changed to naphthalene-2-boronic acid pinacol ester in the synthesis process. Then, purification was performed by extraction using tetrahydrofuran. It was confirmed by Tof-MS and ¹H-NMR that the resulting compound was a disubstituted product, i.e., PXX-(Nap)₂.

### Example 4

Example 4 also relates to dioxaanthanthrene compounds according to the first and second embodiments of the present invention, and further relates to dioxaanthanthrene compounds according to the third and seventh embodiments. A dioxaanthanthrene compound of Example 4 is 3,9-bis(2,2′-bithiophen-5-yl)-peri-xanthenoxanthene [PXX-(BT)₂] represented by structural formula (6) below. That is, R₃ and R₉ are each an aromatic heterocycle (specifically, 2,2′-bithiophen-5-yl group).

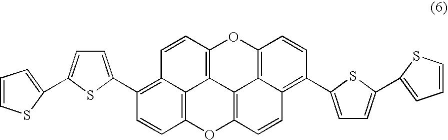

Furthermore, a dioxaanthanthrene compound of Example 4 is 3,9-bis(2,2′-bithiophen-5-yl)-peri-xanthenoxanthene obtained by reacting peri-xanthenoxanthene with bromine to produce 3,9-dibromo-peri-xanthenoxanthene, and then by replacing bromine atoms with 2,2′-bithiophen-5-yl groups.

PXX-(BT)₂ of Example 4 was obtained according to the same scheme as that in Example 1, except that 4,4,5,5-tetramethyl-1,3,2-dioxaborolan-2-yl)benzene was changed to 2,2′-bithiophene-5-boronic acid pinacol ester in the synthesis process. Then, purification was performed by extraction using tetrahydrofuran. It was confirmed by Tof-MS and ¹H-NMR that the resulting compound was a disubstituted product, i.e., PXX-(BT)₂.

### Example 5

Example 5 also relates to dioxaanthanthrene compounds according to the first and second embodiments of the present invention, and further relates to dioxaanthanthrene compounds according to the third and eighth embodiments. A dioxaanthanthrene compound of Example 5 is 3,9-bis(trans-2-(4-pentylphenyl)vinyl)-peri-xanthenoxanthene [PXX-(VPC5)₂] represented by structural formula (7) below. That is, R₃ and R₉ each include a vinyl group, a phenyl group, and an alkyl group.

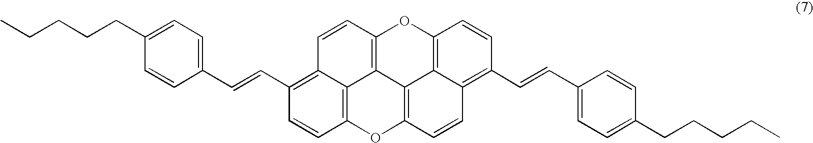

Furthermore, a dioxaanthanthrene compound of Example 5 is 3,9-bis(trans-2-(4-pentylphenyl)vinyl)-peri-xanthenoxanthene obtained by reacting peri-xanthenoxanthene with bromine to produce 3,9-dibromo-peri-xanthenoxanthene, and then by replacing bromine atoms with trans-2-(4-pentylphenyl)vinyl groups.

PXX-(VPC5)₂ of Example 5 was obtained according to the same scheme as that in Example 1, except that 4,4,5,5-tetramethyl-1,3,2-dioxaborolan-2-yl)benzene was changed to 2-[2-(4-pentylphenyl)vinyl]-4,4,5,5-tetramethyl-1,3,2-dioxaborane in the synthesis process. Then, purification was performed by extraction using tetrahydrofuran. It was confirmed by Tof-MS and ¹H-NMR that the resulting compound was a disubstituted product, i.e., PXX-(VPC5)₂.

### Example 6

Example 6 also relates to dioxaanthanthrene compounds according to the first and second embodiments of the present invention, and further relates to dioxaanthanthrene compounds according to the third and ninth embodiments. A dioxaanthanthrene compound of Example 6 is 3,9-di(p-tolyl)-peri-xanthenoxanthene [PXX-(C1Ph)₂] represented by structural formula (8) below. That is, R₃ and R₉ are each an aryl-alkyl group (aryl group partially substituted by an alkyl group; hereinafter, the same).

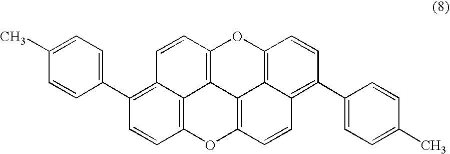

Furthermore, a dioxaanthanthrene compound of Example 6 is 3,9-di(p-tolyl)-peri-xanthenoxanthene obtained by reacting peri-xanthenoxanthene with bromine to produce 3,9-dibromo-peri-xanthenoxanthene, and then by replacing bromine atoms with p-tolyl groups.

PXX-(C1Ph)₂ of Example 6 was obtained according to the same scheme as that in Example 1, except that 4,4,5,5-tetramethyl-1,3,2-dioxaborolan-2-yl)benzene was changed to p-tolylboronic acid in the synthesis process. Then, purification was performed by sublimation under high vacuum, followed by extraction using tetrahydrofuran. It was confirmed by Tof-MS and ¹H-NMR that the resulting compound was a disubstituted product, i.e., PXX-(C1Ph)₂.

### Example 7

Example 7 also relates to dioxaanthanthrene compounds according to the first and second embodiments of the present invention, and further relates to dioxaanthanthrene compounds according to the third and tenth embodiments. A dioxaanthanthrene compound of Example 7 is 3,9-bis(p-ethylphenyl)-peri-xanthenoxanthene [PXX-(C2Ph)₂] represented by structural formula (9) below. That is, R₃ and R₉ are each an aryl-alkyl group.

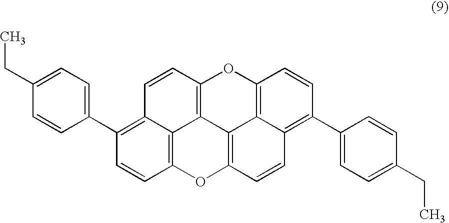

Furthermore, a dioxaanthanthrene compound of Example 7 is 3,9-bis(p-ethylphenyl)-peri-xanthenoxanthene obtained by reacting peri-xanthenoxanthene with bromine to produce 3,9-dibromo-peri-xanthenoxanthene, and then by replacing bromine atoms with p-ethylphenyl groups.

PXX-(C2Ph)₂ of Example 7 was obtained according to the same scheme as that in Example 1, except that 4,4,5,5-tetramethyl-1,3,2-dioxaborolan-2-yl)benzene was changed to p-ethylphenylboronic acid in the synthesis process. Then, purification was performed by sublimation under high vacuum, followed by recrystallization using toluene. It was confirmed by Tof-MS and ¹H-NMR that the resulting compound was a disubstituted product, i.e., PXX-(C2Ph)₂.

### Example 8

Example 8 also relates to dioxaanthanthrene compounds according to the first and second embodiments of the present invention, and further relates to dioxaanthanthrene compounds according to the third and eleventh embodiments. A dioxaanthanthrene compound of Example 8 is 3,9-bis(p-isopropylphenyl)-peri-xanthenoxanthene [PXX-(iC3Ph)₂] represented by structural formula (10) below. That is, R₃ and R₉ are each an aryl-alkyl group.

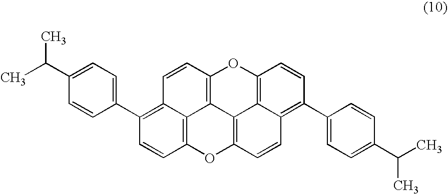

Furthermore, a dioxaanthanthrene compound of Example 8 is 3,9-bis(p-isopropylphenyl)-peri-xanthenoxanthene obtained by reacting peri-xanthenoxanthene with bromine to produce 3,9-dibromo-peri-xanthenoxanthene, and then by replacing bromine atoms with p-isopropylphenyl groups.

PXX-(iC3Ph)₂ of Example 8 was obtained according to the same scheme as that in Example 1, except that 4,4,5,5-tetramethyl-1,3,2-dioxaborolan-2-yl)benzene was changed to p-isopropylphenylboronic acid in the synthesis process. Then, purification was performed by sublimation under high vacuum, followed by recrystallization using toluene. It was confirmed by Tof-MS and ¹H-NMR that the resulting compound was a disubstituted product, i.e., PXX-(iC3Ph)₂.

### Example 9

Example 9 also relates to dioxaanthanthrene compounds according to the first and second embodiments of the present invention, and further relates to dioxaanthanthrene compounds according to the third and twelfth embodiments. A dioxaanthanthrene compound of Example 9 is 3,9-bis(4-propylphenyl)-peri-xanthenoxanthene [PXX-(C3Ph)₂] represented by structural formula (11) below. That is, R₃ and R₉ are each an aryl-alkyl group.

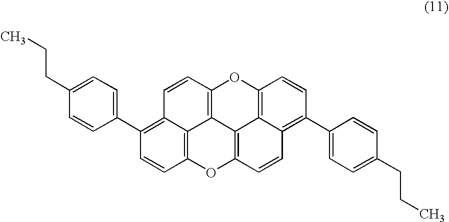

Furthermore, a dioxaanthanthrene compound of Example 9 is 3,9-bis(4-propylphenyl)-peri-xanthenoxanthene obtained by reacting peri-xanthenoxanthene with bromine to produce 3,9-dibromo-peri-xanthenoxanthene, and then by replacing bromine atoms with 4-propylphenyl groups.

PXX-(C3Ph)₂ of Example 9 was obtained according to the same scheme as that in Example 1, except that 4,4,5,5-tetramethyl-1,3,2-dioxaborolan-2-yl)benzene was changed to 4-propylphenylboronic acid in the synthesis process. Then, purification was performed by sublimation under high vacuum, followed by recrystallization using toluene. It was confirmed by Tof-MS and ¹H-NMR that the resulting compound was a disubstituted product, i.e., PXX-(C3Ph)₂.

### Example 10

Example 10 also relates to dioxaanthanthrene compounds according to the first and second embodiments of the present invention, and further relates to dioxaanthanthrene compounds according to the third and thirteenth embodiments.

A dioxaanthanthrene compound of Example 10 is 3,9-bis(4-butylphenyl)-peri-xanthenoxanthene [PXX-(C4Ph)₂] represented by structural formula (12) below. That is, R₃ and R₉ are each an aryl-alkyl group.

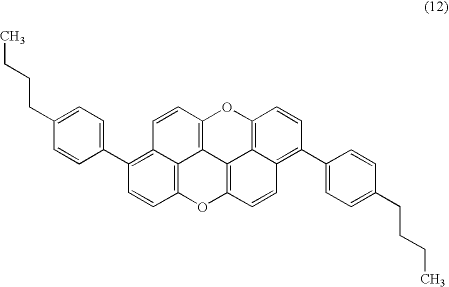

Furthermore, a dioxaanthanthrene compound of Example 10 is 3,9-bis(4-butylphenyl)-peri-xanthenoxanthene obtained by reacting peri-xanthenoxanthene with bromine to produce 3,9-dibromo-peri-xanthenoxanthene, and then by replacing bromine atoms with 4-butylphenyl groups.

PXX-(C4Ph)₂ of Example 10 was obtained according to the same scheme as that in Example 1, except that 4,4,5,5-tetramethyl-1,3,2-dioxaborolan-2-yl)benzene was changed to 4-butylphenylboronic acid in the synthesis process. Then, purification was performed by sublimation under high vacuum, followed by recrystallization using toluene. It was confirmed by Tof-MS and ¹H-NMR that the resulting compound was a disubstituted product, i.e., PXX-(C4Ph)₂.

### Example 11

Example 11 also relates to dioxaanthanthrene compounds according to the first and second embodiments of the present invention, and further relates to dioxaanthanthrene compounds according to the third and fourteenth embodiments. A dioxaanthanthrene compound of Example 11 is 3,9-bis(4-nonylphenyl)-peri-xanthenoxanthene [PXX-(C9Ph)₂] represented by structural formula (13) below. That is, R₃ and R₉ are each an aryl-alkyl group.

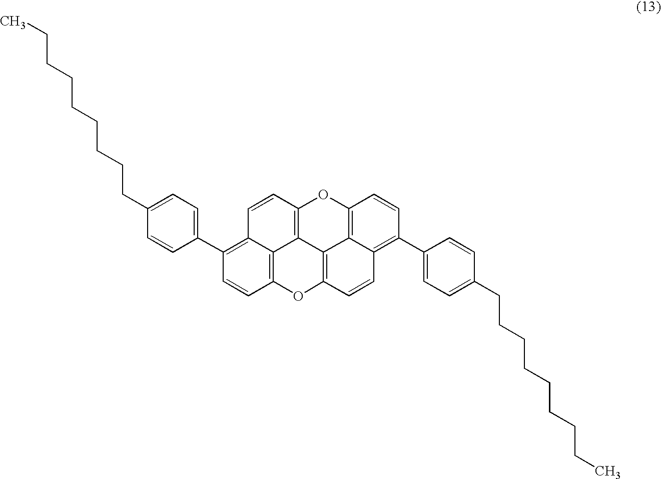

Furthermore, a dioxaanthanthrene compound of Example 11 is 3,9-bis(4-nonylphenyl)-peri-xanthenoxanthene obtained by reacting peri-xanthenoxanthene with bromine to produce 3,9-dibromo-peri-xanthenoxanthene, and then by replacing bromine atoms with 4-nonylphenyl groups.

PXX-(C9Ph)₂ of Example 11 was obtained according to the same scheme as that in Example 1, except that 4,4,5,5-tetramethyl-1,3,2-dioxaborolan-2-yl)benzene was changed to 4-normal-nonylbenzene boronic acid in the synthesis process. Then, purification was performed by sublimation under high vacuum, followed by recrystallization using toluene. It was confirmed by Tof-MS that the resulting compound was a disubstituted product, i.e., PXX-(C9Ph)₂.

### Example 12

Example 12 also relates to dioxaanthanthrene compounds according to the first and second embodiments of the present invention, and further relates to dioxaanthanthrene compounds according to the third and fifteenth embodiments. A dioxaanthanthrene compound of Example 12 is 3,9-bis(p-biphenyl)-peri-xanthenoxanthene [PXX-(BPh)₂] represented by structural formula (14) below. That is, R₃ and R₉ are each an aryl group.

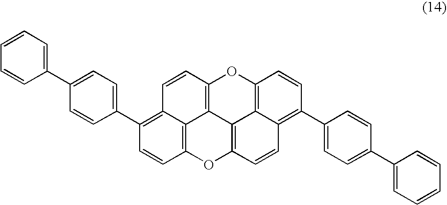

Furthermore, a dioxaanthanthrene compound of Example 12 is 3,9-bis(p-biphenyl)-peri-xanthenoxanthene obtained by reacting peri-xanthenoxanthene with bromine to produce 3,9-dibromo-peri-xanthenoxanthene, and then by replacing bromine atoms with p-biphenyl groups.

PXX-(BPh)₂ of Example 12 was obtained according to the same scheme as that in Example 1, except that 4,4,5,5-tetramethyl-1,3,2-dioxaborolan-2-yl)benzene was changed to 4-biphenylboronic acid in the synthesis process. Then, purification was performed by sublimation under high vacuum, followed by extraction using benzene. It was confirmed by Tof-MS that the resulting compound was a disubstituted product, i.e., PXX-(BPh)₂.

### Example 13

Example 13 relates to semiconductor devices according to the first and second embodiments of the present invention. A semiconductor device (specifically, field-effect transistor, FET) of Example 13 includes a gate electrode, a gate insulating layer, source/drain electrodes, and a channel-forming region that are disposed on a base, in which the channel-forming region is composed of the dioxaanthanthrene compound represented by structural formula

(1) described above, wherein at least one of R₃ and R₉ represents a substituent other than hydrogen. Furthermore, a semiconductor device of Example 13 includes a gate electrode, a gate insulating layer, source/drain electrodes, and a channel-forming region that are disposed on a base, in which the channel-forming region is composed of the dioxaanthanthrene compound represented by structural formula
(2) described above, wherein at least one of R₁, R₃, R₄, R₅, R₇, R₉, R₁₀, and R₁₁ represents a substituent other than hydrogen.

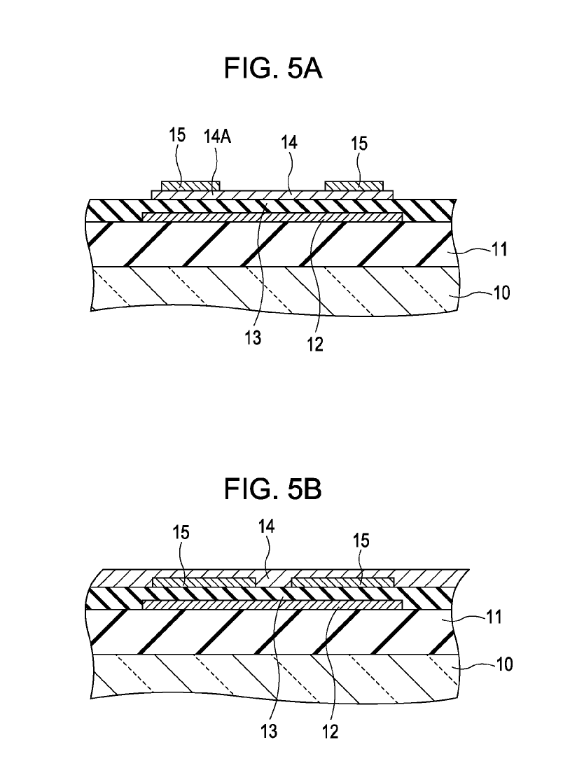

More specifically, a semiconductor device of Example 13 is a bottom gate/top contact type FET, a schematic partial sectional view of which is shown in FIG. 5A, and includes

(A) a gate electrode 12 disposed on a base (10, 11), (B) a gate insulating layer 13 disposed on the gate electrode 12,
(C) a channel-forming region 14 and a channel-forming region extension 14A disposed on the gate insulating layer 13, and
(D) source/drain electrodes 15 disposed on the channel-forming region extension 14A.

The base (10, 11) includes a substrate 10 composed of a glass substrate and an insulating film 11 composed of SiO₂ disposed on the surface thereof. Each of the gate electrode 12 and the source/drain electrodes 15 is composed of a gold thin film. The gate insulating layer 13 is composed of SiO₂. Each of the channel-forming region 14 and the channel-forming region extension 14A is composed of any one of the dioxaanthanthrene compounds described in Examples 1 to 12. The gate electrode 12 and the gate insulating layer 13 are more specifically disposed on the insulating film 11.

An outline of a method for fabricating the bottom gate/top contact type FET (specifically, TFT) will be described below.

[Step-1300A]

First, a gate electrode 12 is formed on a base (which includes a glass substrate 10 and an insulating film 11 composed of SiO₂ disposed on the surface thereof). Specifically, a resist layer (not shown) is formed on the insulating film 11 using a lithographic technique, the resist layer having an opening corresponding to the portion at which the gate electrode 12 is to be formed. Next, a chromium (Cr) layer (not shown) as an adhesion layer and a gold (Au) layer as the gate electrode 12 are formed in that order by a vacuum deposition method over the entire surface, and then the resist layer is removed. Thereby, the gate electrode 12 can be obtained by a lift-off method.

[Step-1310A]

Next, a gate insulating layer 13 is formed on the base (insulating film 11) including the gate electrode 12. Specifically, the gate insulating layer 13 composed of SiO₂ is formed by sputtering over the gate electrode 12 and the insulating film 11. In the process of forming the gate insulating layer 13, by covering part of the gate electrode 12 with a hard mask, a lead portion (not shown) of the gate electrode 12 can be formed without performing a photolithographic process.

[Step-1320A]

Next, a channel-forming region 14 and a channel-forming region extension 14A are formed on the gate insulating layer 13. Specifically, using a vacuum deposition method, any one of the dioxaanthanthrene compounds described in Examples 1 to 12 is deposited.

[Step-1330A]

Then, source/drain electrodes 15 are formed on the channel-forming region extension 14A so as to sandwich the channel-forming region 14. Specifically, a chromium (Cr) layer (not shown) as an adhesion layer and gold (Au) layers as the source/drain electrodes 15 are formed in that order by a vacuum deposition method over the entire surface. Thereby, the structure shown in FIG. 5A can be obtained. In the process of forming the source/drain electrodes 15, by covering part of the channel-forming region extension 14A with a hard mask, the source/drain electrodes 15 can be formed without performing a photolithographic process.

[Step-1340A]

Lastly, an insulating layer (not shown) which is a passivation film is formed over the entire surface, and openings are formed in the insulating layer on top of the source/drain electrodes 15. After a wiring material layer is formed over the entire surface including the inside of the openings, the wiring material layer is subjected to patterning. Thereby, a bottom gate/top contact type FET (TFT) in which interconnect lines (not shown) connected to the source/drain electrodes 15 are formed on the insulating layer can be obtained.

The FET is not limited to the bottom gate/top contact type FET shown in FIG. 5A, and may be a bottom gate/bottom contact type FET, a top gate/top contact type FET, or a top gate/bottom contact type FET.

A bottom gate/bottom contact type FET, a schematic partial sectional view of which is shown in FIG. 5B, includes (A) a gate electrode 12 disposed on a base (10, 11), (B) a gate insulating layer 13 disposed on the gate electrode 12, (C) source/drain electrodes 15 disposed on the gate insulating layer 13; and (D) a channel-forming region 14 disposed between the source/drain electrodes 15 and on the gate insulating layer 13.

An outline of a method for fabricating the bottom gate/bottom contact type TFT will be described below.

[Step-1300B]

First, a gate electrode 12 is formed on a base (insulating film 11) as in Step-1300A, and then a gate insulating layer 13 is formed over the gate electrode 12 and the insulating film 11 as in Step-1310A.

[Step-1310B]

Next, source/drain electrodes 15 composed of gold (Au) layers are formed on the gate insulating layer 13. Specifically, a resist layer is formed on the gate insulating layer 13 using a lithographic technique, the resist layer having openings corresponding to the portions at which the source/drain electrodes 15 are to be formed. Next, as in Step-1300A, a chromium (Cr) layer (not shown) as an adhesion layer and gold (Au) layers as the source/drain electrodes 15 are formed in that order by a vacuum deposition method over the resist layer and the gate insulating layer 13, and then the resist layer is removed. Thereby, the source/drain electrodes 15 can be obtained by a lift-off method.

[Step-1320B]

Then, as in Step-1320A, a channel-forming region 14 is formed between the source/drain electrodes 15 and on the gate insulating layer 13. Thereby, the structure shown in FIG. 5B can be obtained.

[Step-1330B]

Lastly, by carrying out the same step as Step-1340A, a bottom gate/bottom contact type FET (TFT) can be obtained.

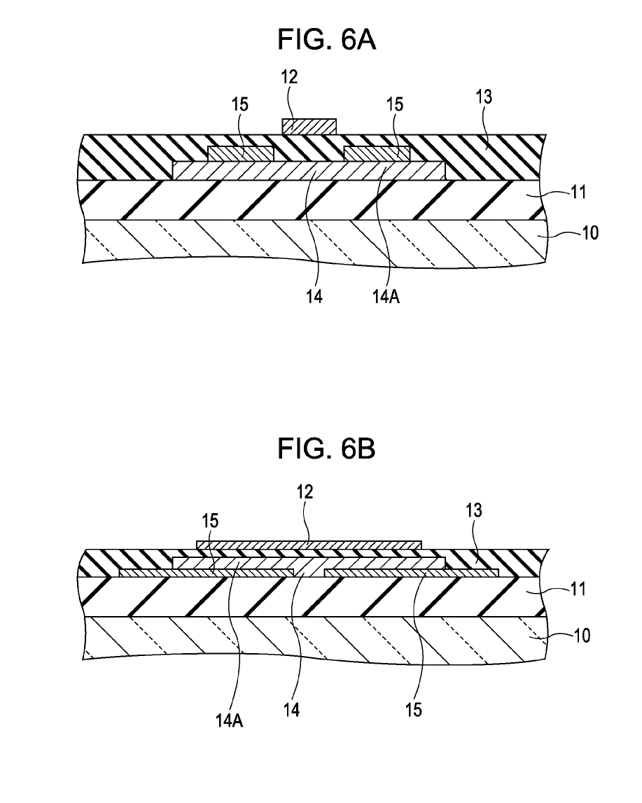

A top gate/top contact type FET, a schematic partial sectional view of which is shown in FIG. 6A, includes (A) a channel-forming region 14 and a channel-forming region extension 14A disposed on a base (10, 11), (B) source/drain electrodes 15 disposed on the channel-forming region extension 14A, (C) a gate insulating layer 13 disposed on the source/drain electrodes 15 and the channel-forming region 14, and (D) a gate electrode 12 disposed on the gate insulating layer 13.

An outline of a method for fabricating the top gate/top contact type TFT will be described below.

[Step-1300C]

First, a channel-forming region 14 and a channel-forming region extension 14A are formed, using the same method as that in Step-1320A, on a base (including a glass substrate 10 and an insulating film 11 composed of SiO₂ disposed on the surface thereof). [Step-1310C]

Next, source/drain electrodes 15 are formed on the channel-forming region extension 14A so as to sandwich the channel-forming region 14. Specifically, a chromium (Cr) layer (not shown) as an adhesion layer and a gold (Au) layer as source/drain electrodes 15 are formed in that order by a vacuum deposition method over the entire surface. In the process of forming the source/drain electrodes 15, by covering part of the channel-forming region extension 14A with a hard mask, the source/drain electrodes 15 can be formed without performing a photolithographic process.

[Step-1320C]

Next, a gate insulating layer 13 is formed over the source/drain electrodes 15 and the channel-forming region 14. Specifically, by applying PVA by spin coating over the entire surface, the gate insulating layer 13 can be obtained.

[Step-1330C]

Then, a gate electrode 12 is formed on the gate insulating layer 13. Specifically, a chromium (Cr) layer (not shown) as an adhesion layer and a gold (Au) layer as the gate electrode 12 are formed in that order by a vacuum deposition method over the entire surface. Thereby, the structure shown in FIG. 6A can be obtained. In the process of forming the gate electrode 12, by covering part of the gate insulating layer 13 with a hard mask, the gate electrode 12 can be formed without performing a photolithographic process. Lastly, by carrying out the same step as Step-1340A, a top gate/top contact type FET (TFT) can be obtained.

A top gate/bottom contact type FET, a schematic partial sectional view of which is shown in FIG. 6B, includes (A) source/drain electrodes 15 disposed on a base (10, 11), (B) a channel-forming region 14 disposed between the source/drain electrodes 15 and on the base (10, 11), (C) a gate insulating layer 13 disposed on the channel-forming region 14, and (D) a gate electrode 12 disposed on the gate insulating layer 13.

An outline of a method for fabricating the top gate/bottom contact type TFT will be described below.

[Step-1300D]

First, source/drain electrodes 15 are formed on a base (which includes a glass substrate 10 and an insulating film 11 composed of SiO₂ disposed on the surface thereof). Specifically, a chromium (Cr) layer (not shown) as an adhesion layer and a gold (Au) layer as the source/drain electrodes 15 are formed by a vacuum deposition method on the insulating film 11. In the process of forming the source/drain electrodes 15, by covering part of the base (insulating film 11) with a hard mask, the source/drain electrodes 15 can be formed without performing a photolithographic process.

[Step-1310D]

Then, a channel-forming region 14 is formed between the source/drain electrodes 15 and on the base (insulating film 11) using the same method as that in Step-1320A. A channel-forming region extension 14A is actually formed on the source/drain electrodes 15.

[Step-1320D]

Next, a gate insulating layer 13 is formed over the source/drain electrodes 15 and the channel-forming region 14 (more specifically, over the channel-forming region 14 and the channel-forming region extension 14A) as in Step-1320C.

[Step-1330D]

Then, a gate electrode 12 is formed on the gate insulating layer 13 as in Step-1330C. Thereby, the structure shown in FIG. 6B can be obtained. Lastly, by carrying out the same step as Step-1340A, a top gate/bottom contact type FET (TFT) can be obtained.

The present invention has been described on the basis of the preferred Examples. However, the present invention is not limited to these Examples. The configurations and structures of the semiconductor devices, fabrication conditions, and the fabrication methods described above are merely exemplification, and can be altered appropriately. When the semiconductor devices according to the embodiments of the present invention are applied to or used for display devices or various types of electronic apparatuses, monolithic integrated circuits in which many FETs are integrated on supports or supporting members may be fabricated, or the individual FETs may be separated by cutting to produce discrete components.

The present application contains subject matter related to that disclosed in Japanese Priority Patent Application JP 2008-136292 filed in the Japan Patent Office on May 26, 2008, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A dioxaanthanthrene compound represented by structural formula (1): 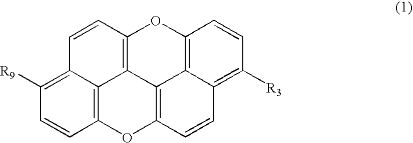
wherein at least one of R₃ and R₉ represents a substituent other than hydrogen.

2. The dioxaanthanthrene compound according to claim 1, wherein the substituent other than hydrogen is a substituent selected from the group consisting of an alkyl group, a cycloalkyl group, an alkenyl group, an alkynyl group, an aryl group, an aryl-alkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorohydrocarbon group, a cyano group, a nitro group, a hydroxy group, a mercapto group, and a silyl group.

3. The dioxaanthanthrene compound according to claim 1, wherein the substituent other than hydrogen is a substituent selected from the group consisting of an alkyl group, an alkenyl group, an aryl group, an aryl-alkyl group, an aromatic heterocycle, and a halogen atom.

4. A dioxaanthanthrene compound represented by structural formula (2): 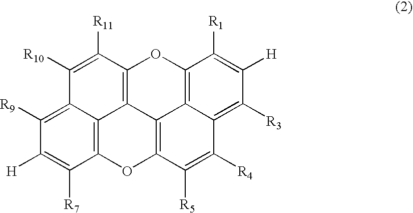
wherein at least one of R₁, R₃, R₄, R₅, R₇, R₉, R₁₀, and R₁₁ represents a substituent other than hydrogen.

5. The dioxaanthanthrene compound according to claim 4, wherein at least one of R₃ and R₉ is a substituent other than hydrogen, and at least one of R₁, R₄, R₅, R₇, R₁₀, and R₁₁ is a substituent other than hydrogen.

6. The dioxaanthanthrene compound according to claim 4, wherein the substituent other than hydrogen is a substituent selected from the group consisting of an alkyl group, a cycloalkyl group, an alkenyl group, an alkynyl group, an aryl group, an aryl-alkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorohydrocarbon group, a cyano group, a nitro group, a hydroxy group, a mercapto group, and a silyl group.

7. The dioxaanthanthrene compound according to claim 4, wherein the substituent other than hydrogen is a substituent selected from the group consisting of an alkyl group, an alkenyl group, an aryl group, an aryl-alkyl group, an aromatic heterocycle, and a halogen atom.

8. A semiconductor device comprising:
a gate electrode;
a gate insulating layer;
source/drain electrodes; and
a channel-forming region,
the gate electrode, the gate insulating layer, the source/drain electrodes, and the channel-forming region being disposed on a base,
wherein the channel-forming region is composed of a dioxaanthanthrene compound represented by structural formula (1): 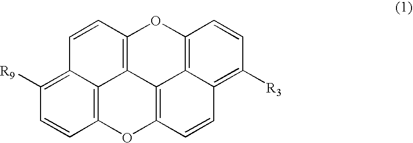
wherein at least one of R₃ and R₉ represents a substituent other than hydrogen.

9. The semiconductor device according to claim 8, wherein the substituent other than hydrogen is a substituent selected from the group consisting of an alkyl group, a cycloalkyl group, an alkenyl group, an alkynyl group, an aryl group, an aryl-alkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorohydrocarbon group, a cyano group, a nitro group, a hydroxy group, a mercapto group, and a silyl group.

10. The semiconductor device according to claim 8, wherein the substituent other than hydrogen is a substituent selected from the group consisting of an alkyl group, an alkenyl group, an aryl group, an aryl-alkyl group, an aromatic heterocycle, and a halogen atom.

11. A semiconductor device comprising:
a gate electrode;
a gate insulating layer;
source/drain electrodes; and
a channel-forming region,
the gate electrode, the gate insulating layer, the source/drain electrodes, and the channel-forming region being disposed on a base,
wherein the channel-forming region is composed of a dioxaanthanthrene compound represented by structural formula (2): 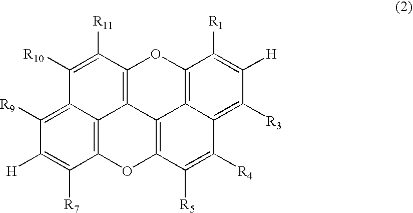
wherein at least one of R₁, R₃, R₄, R₅, R₇, R₉, R₁₀, and R₁₁ represents a substituent other than hydrogen.

12. The semiconductor device according to claim 11, wherein at least one of R₃ and R₉ is a substituent other than hydrogen, and at least one of R₁, R₄, R₅, R₇, R₁₀, and R₁₁ is a substituent other than hydrogen.

13. The semiconductor device according to claim 11, wherein the substituent other than hydrogen is a substituent selected from the group consisting of an alkyl group, a cycloalkyl group, an alkenyl group, an alkynyl group, an aryl group, an aryl-alkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorohydrocarbon group, a cyano group, a nitro group, a hydroxy group, a mercapto group, and a silyl group.

14. The semiconductor device according to claim 11, wherein the substituent other than hydrogen is a substituent selected from the group consisting of an alkyl group, an alkenyl group, an aryl group, an aryl-alkyl group, an aromatic heterocycle, and a halogen atom.

15. A dioxaanthanthrene compound comprising 6,12-dioxaanthanthrene which is replaced at least one of positions 3 and 9 with a substituent other than hydrogen, the dioxaanthanthrene compound being obtained by halogenating peri-xanthenoxanthene into 3,9-dihalo-peri-xanthenoxanthene and then replacing the halogen atom with the substituent.

16. The dioxaanthanthrene compound according to claim 15, wherein the halogen atom is bromine.

17. The dioxaanthanthrene compound according to claim 15 or 16, wherein the substituent is an aryl group or an aryl-alkyl group.

18. The dioxaanthanthrene compound according to claim 15 or 16, wherein the substituent is an aryl group which is replaced at least one of positions 2 to 6 with an alkyl group or an aryl group.

19. The dioxaanthanthrene compound according to claim 15 or 16, wherein the substituent is a p-tolyl group, p-ethylphenyl group, p-isopropylphenyl group, 4-propylphenyl group, 4-butylphenyl group, 4-nonylphenyl group, or p-biphenyl.

20. A dioxaanthanthrene compound comprising 3,9-diphenyl-peri-xanthenoxanthene obtained by reacting peri-xanthenoxanthene with bromine to produce 3,9-dibromo-peri-xanthenoxanthene, and then by replacing bromine atoms with phenyl groups.

21. A dioxaanthanthrene compound comprising 3,9-di(trans-1-octen-1-yl)-peri-xanthenoxanthene obtained by reacting peri-xanthenoxanthene with bromine to produce 3,9-dibromo-peri-xanthenoxanthene, and then by replacing bromine atoms with trans-1-octen-1-yl groups.

22. A dioxaanthanthrene compound comprising 3,9-di(2-naphthyl)-peri-xanthenoxanthene obtained by reacting peri-xanthenoxanthene with bromine to produce 3,9-dibromo-peri-xanthenoxanthene, and then by replacing bromine atoms with β-naphthyl groups.

23. A dioxaanthanthrene compound comprising 3,9-bis(2,2′-bithiophen-5-yl)-peri-xanthenoxanthene obtained by reacting peri-xanthenoxanthene with bromine to produce 3,9-dibromo-peri-xanthenoxanthene, and then by replacing bromine atoms with 2,2′-bithiophen-5-yl groups.

24. A dioxaanthanthrene compound comprising 3,9-bis(trans-2-(4-pentylphenyl)vinyl)-peri-xanthenoxanthene obtained by reacting peri-xanthenoxanthene with bromine to produce 3,9-dibromo-peri-xanthenoxanthene, and then by replacing bromine atoms with trans-2-(4-pentylphenyl)vinyl groups.

25. A dioxaanthanthrene compound comprising 3,9-di(p-tolyl)-peri-xanthenoxanthene obtained by reacting peri-xanthenoxanthene with bromine to produce 3,9-dibromo-peri-xanthenoxanthene, and then by replacing bromine atoms with p-tolyl groups.

26. A dioxaanthanthrene compound comprising 3,9-bis(p-ethylphenyl)-peri-xanthenoxanthene obtained by reacting peri-xanthenoxanthene with bromine to produce 3,9-dibromo-peri-xanthenoxanthene, and then by replacing bromine atoms with p-ethylphenyl groups.

27. A dioxaanthanthrene compound comprising 3,9-bis(p-isopropylphenyl)-peri-xanthenoxanthene obtained by reacting peri-xanthenoxanthene with bromine to produce 3,9-dibromo-peri-xanthenoxanthene, and then by replacing bromine atoms with p-isopropylphenyl groups.

28. A dioxaanthanthrene compound comprising 3,9-bis(4-propylphenyl)-peri-xanthenoxanthene obtained by reacting peri-xanthenoxanthene with bromine to produce 3,9-dibromo-peri-xanthenoxanthene, and then by replacing bromine atoms with 4-propylphenyl groups.

29. A dioxaanthanthrene compound comprising 3,9-bis(4-butylphenyl)-peri-xanthenoxanthene obtained by reacting peri-xanthenoxanthene with bromine to produce 3,9-dibromo-peri-xanthenoxanthene, and then by replacing bromine atoms with 4-butylphenyl groups.

30. A dioxaanthanthrene compound comprising 3,9-bis(4-nonylphenyl)-peri-xanthenoxanthene obtained by reacting peri-xanthenoxanthene with bromine to produce 3,9-dibromo-peri-xanthenoxanthene, and then by replacing bromine atoms with 4-nonylphenyl groups.

31. A dioxaanthanthrene compound comprising 3,9-bis(p-biphenyl)-peri-xanthenoxanthene obtained by reacting peri-xanthenoxanthene with bromine to produce 3,9-dibromo-peri-xanthenoxanthene, and then by replacing bromine atoms with p-biphenyl groups.
